(19) United States
(12) Patent Application Publication
(10) Pub. No.: US 2003/0113817 A1
(43) Pub. Date: Jun. 19, 2003

(54) Hemogen-EDAG: novel nuclear factors expressed in hematopoietic development

(76) Inventors: Li Li, Detroit, MI (US); Li Yang, Windsor (CA)

Correspondence Address: Venable, P.O. Box 34385, Washington, DC, 20043-9998, US

(21) Appl. No.: 10/103,140

(22) Filed: Mar. 22, 2002

Related U.S. Application Data
(60) Provisional application No. 60/277,624, filed on Mar. 22, 2001.

(51) Int. Cl. C12Q 1/68; G01N 33/567; C07H 21/04; C12N 9/00; C12P 21/02; C07K 16/40; C12N 5/08

(52) U.S. Cl. 435/7.21; 435/69.1; 435/320.1; 435/372; 536/23.2; 435/6; 435/183; 530/388.26

(57) ABSTRACT

A novel murine gene, designated Hemogen (hemopoietic gene) is sequentially expressed in active hematopoietic sites and downregulated during blood cell differentiation. Hemogen is a nuclear protein. A human homologue of Hemogen, named EDAG, which maps to chromosome 9q22, a leukemia breakpoint, exhibits also specific expression in hematopoietic tissues and cells. Hemogen and EDAG play an important role in hematopoietic development and neoplasms.

### STATEMENT OF RIGHTS TO INVENTIONS MADE UNDER FEDERALLY SPONSORED RESEARCH

This invention was funded in part by a grant from the National Heart Lung and Blood Institute (HL 58916-01A1), National Institutes of Health which provides to the United States government certain rights in this invention.

### BACKGROUND OF THE INVENTION

1. Field of the Invention

. The present invention in the field of medicine and molecular biology is directed to new nucleic acid encoding a protein that is involved in early stages of hematopoiesis. The gene maps to a chromosomal region rich in translocation breaks associated with human hematological disease, primarily leukemias.

2. Description of the Background Art

References cited herein are listed before the claims.

Hematopoiesis is a dynamic process with sequential shifting of primary hematopoietic sites from yolk sac to fetal liver and finally bone marrow (BM). During embryonic development, hematopoietic tissues are derived from the ventral mesoderm. The first blood cells, primitive erythrocytes, appear in the blood islands in extraembryonic yolk sac at about embryonic day 7.5 (E7.5) in mice. By E12, fetal liver becomes the predominant site of blood cell formation in the embryo (Dzierzak and Medvinsky, 1995). Just prior to birth and thereafter, hematopoiesis in fetal liver gradually decreases, and BM becomes the major hematopoietic site. In contrast to the human spleen, in mouse spleen, hematopoiesis, particularly erythropoiesis is very active in the red pulp in adulthood (Seifert and Marks, 1985). Hematopoiesis in the yolk sac is termed “primitive” (embryonic) hematopoiesis, and that in the fetal liver and BM is termed “definitive” (adult) hematopoiesis. Mature blood cells of the three main hematopoietic lineages, erythroid, myeloid and lymphoid, are derived from common pluripotent hematopoietic stem cells (Spangrude et al., 1988). The study of hematopoiesis has been facilitated by the identification of a variety of regulatory genes important for hematopoietic induction, lineage selection and blood cell differentiation (Engel and Murre, 1999; Orkin, 1995; Sieweke and Graf, 1998). Mutations or translocations in many of these genes are important in hematopoietic malignancies (Rowley, 1998; Sawyers, 1998).

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present inventors cloned a novel murine gene, designated Hemogen (hemopoietic gene), which was sequentially expressed in active hematopoietic sites and downregulated in the process of blood cell differentiation. Hemogen transcripts were specifically detected in blood islands, primitive blood cells and fetal liver during embryogenesis, and then remained in bone marrow and spleen in adult mice. Immunostaining demonstrated that Hemogen is a nuclear protein.

The present inventors also discovered a human homologue of Hemogen, named EDAG, which was mapped to chromosome 9q22, a leukemia breakpoint. Like Hemogen, EDAG exhibited specific expression in hematopoietic tissues and cells. Hemogen and EDAG play an important role in hematopoietic development and neoplasms.

### BRIEF DESCRIPTION OF THE DRAWINGS

FIG. 1 presents the nucleotide sequence of mouse Hemogen Numbers on the left refer to the nucleotide sequence (SEQ ID NO:1) (upper) and the deduced amino acid sequence (SEQ ID NO:2) (lower). The first ATG (191-193 nucleotides) defines a 1512 bp ORF with multiple upstream stop codons (upper case and bold) in all three reading frames. The Kozak consensus sequence around ATG (191-193) is underlined. A polyadenylation signal (double-underscored) is 16 nucleotides upstream of the poly(A) tail. The amino acid sequence contains 503 residues with an N-terminal basic domain (underlined) and an acidic domain at the C-terminus (residues 450-480, double underline). The region of residues 34-50 (in brackets) is a predicted coiled-coil domain. This protein also contains a bipartite nuclear localization signal (residues 61-78, underlined italic) that is a basic amino acid cluster.

FIG. 2 is a photomicrograph showing Hemogen protein localized in cell nuclei. The pcDNA3.1 plasmid with Hemogen-FLAG fusion gene was transfected into COS-7 cells and detected with anti-FLAG antibody. The signal was localized in the cell nuclei (nu) but not in the nucleoli (no).

FIGS. 3A-3Q are a series of photomicrographs showing expression of Hemogen during mouse embryogenesis by in situ hybridization. Digoxigenin-labeled antisense RNA probes were hybridized with mouse frontal section at E8.5 (panel A) and sagittal sections at 9.5, 10.5, 11.5, 12.5 and 14.5 (respectively in panels D, F, I, L, and 0). The Hemogen transcripts are shown as purple staining. Panels B, E, G, J, M, and P show circulating blood cells in panels A, D, F, I, L and 0 at high magnification (1000×). The high magnification (1000×) of the blood island is shown in panel C. The high magnification (1000×) of fetal livers are shown in panels H, K, N and Q. Scale bars=1 mm. ao: aorta, bc: blood cell, bi: blood island, lv: liver.

FIGS. 4A-4B is a set of photomicrographs showing E10.5 and E12.5 blood cells. Embryo sections were stained with hematoxylin to show the distinct morphology of circulating blood cells at these two stages. (Panel A). The E10.5 primitive erythrocytes have higher nucleus/cytoplasm ratio. (B). The E12.5 primitive erythrocytes have more condensed nuclei.

FIG. 5A-C shows a Northern blot analysis of Hemogen. Total RNA (˜15 μg) from each indicated tissue was hybridized with a ³²P-labeled antisense RNA probe derived from Hemogen. Panel A: A ˜2.4 kb message was detected in spleen and BM by 5 h exposure. Panel B: The same filter was overexposed for 5 days. Besides the signals in BM and spleen, three very weak bands (arrows) were detected in the peripheral blood but not in other tissues. Panel C: The same agarose gel was stained with ethidium bromide before transfer to monitor RNA loading.

FIGS. 6A-6C is a series of photomicrographs showing expression of Hemogen in adult mouse spleen by in situ hybridization. Panel A: A spleen section was hybridized with the Hemogen sense RNA probes as a negative control. Panel B: By hybridization with antisense RNA probe, Hemogen transcripts were localized in the red pulp (rp) but not in the white pulp (wp). Panel C is a higher magnification (1000×) of the positive-staining cells in the red pulp. Scale bar=1 mm.

FIG. 7 shows expression analysis of Hemogen by RT-PCR. The PCR products were amplified from the templates as indicated. Lanes containing RT-PCR reactions without reverse transcriptase are labeled RT(−). Histone H3 was used as the internal control.

FIG. 8 shows the amino acid sequence alignment of Hemogen, EDAG and RP59. The sequences were aligned by ClustalW program. The mouse gene Hemogen (accession # AF269248) shares 70% and 43% identity with a rat gene RP59 (accession # AJ302650) and a human gene EDAG (accession # AF322875) respectively at the amino acid level. The nuclear localization signal (61-78 residues) and coiled-coil domain (34-50 residues) are highly conserved.

FIGS. 9A-9B shows an expression analysis of EDAG in human tissues, cultured cells and cell lines. Panel A: Northern analysis of human tissue RNA blots with ³²P-labeled EDAG cDNA probes. Two isoforms, a 2.4 kb major isoform and a 1.8 kb minor isoform, were detected (arrow). Panel B shows expression of EDAG in hematopoietic tissues, cultured cells, cell lines and non-hematopoietic cell lines by RT-PCR. Histone H3 was used as the internal control.

### DESCRIPTION OF THE PREFERRED EMBODIMENTS

The present invention provides a new nuclear protein, Hemogen in mice, EDAG in humans (the names are used interchangeably herein), which shows a spatial-temporal expression pattern corresponding to the ontogeny of hematopoiesis. Its expression is strictly localized to hematopoietic tissues from embryonic stages through adulthood. Hemogen is differentially expressed in immature hematopoietic progenitor cells but downregulated in mature nucleated blood cells. A close correlation exists between Hemogen expression and hematopoiesis.

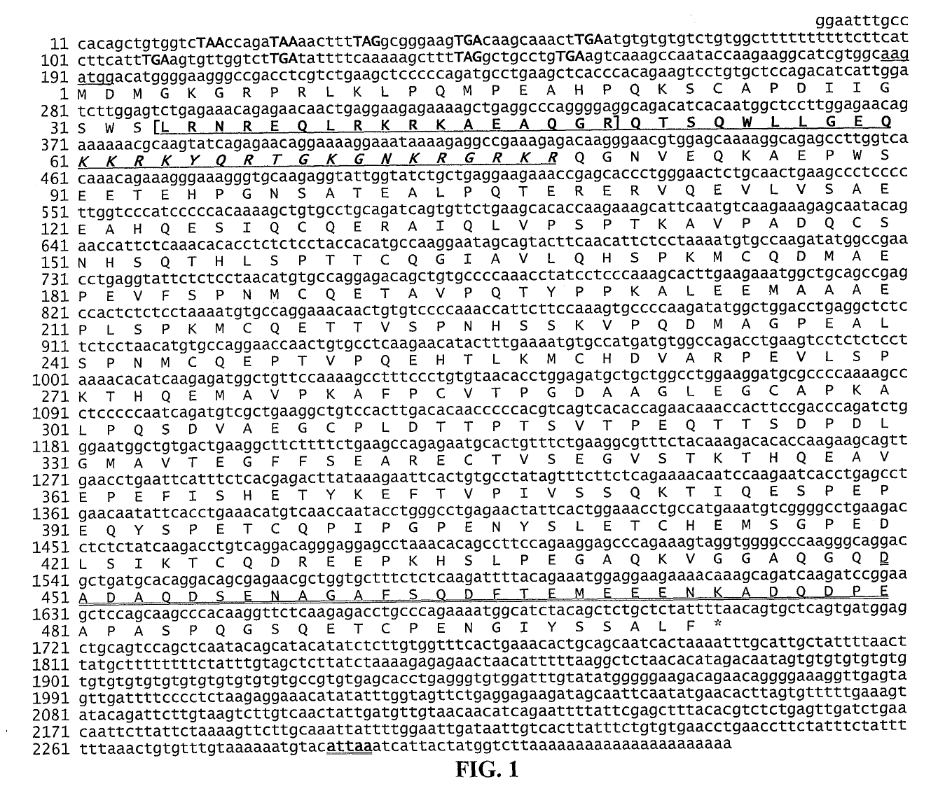

The nucleic acid (SEQ ID NO: 1) and deduced amino acid sequence (SEQ ID NO:2 of Hemogen is shown in FIG. 1

The nucleotide sequence (SEQ ID NO:3) and amino acid sequence (SEQ ID NO:4) of human EDAG are shown below. The nucleotide sequence includes both 5′ and 3′ flanking non-coding sequence as well. Only the nucleotide sequence is numbered

| | |
|---|---|
| 1 | gttatgaagataggtactg |
| 20 | tgggtgttagaaagattcacggcaaaacagggaagcatctaggct |
| 65 | gcttgtggaagtcagaccaaaatagcaggaaggtattgcagcaag |
| 110 | atggatttgggaaaggaccaatctcatttgaagcaccatcagaca |
| | M D L G K D Q S H L K H H Q T |
| 155 | cctgaccctcatcaagaagagaaccattctccagaagtcattgga |
| | P D P H Q E E N H S P E V I G |
| 200 | acctggagtttgagaaacagagaactacttagaaaaagaaaagct |
| | T W S L R N R E L L R K R K A |
| 245 | gaagtgcatgaaaaggaaacatcacaatggctatttggagaacag |
| | E V H E K E T S Q W L F G E Q |
| 290 | aaaaaacgcaagcagcagagaacaggaaaaggaaatcgaagaggc |
| | K K R K Q Q P T G K G N R R G |
| 335 | agaaagagacaacaaaacacagaattgaaggtggagcctcagcca |
| | R K R Q Q N T E L K V E P Q P |
| 380 | cagatagaaaaggaaatagtggagaaagcactggcacctatagag |
| | Q I E K E I V E K A L A P I E |
| 425 | aaaaaaactgagccacctgggagcataaccaaagtatttccttca |
| | K K T E P P G S I T K V F P S |
| 470 | gtagcctccccgcaaaaagttgtgcctgaggaacacttttctgaa |
| | V A S P Q K V V P E E H F S E |
| 515 | atatgtcaagaaagtaacatatatcaggagaatttttctgagtac |
| | I C Q E S N I Y Q E N F S E Y |
| 560 | caagaaatagcagtacaaaaccattcttctgaaacatgccaacat |
| | Q E I A V Q N H S S E T C Q H |
| 605 | gtgtctgaacctgaagacctctctcctaaaatgtaccaagaaata |
| | V S E P E D L S P K M Y Q E I |
| 650 | tctgtacttcaagacaattcttccaaaatatgccaagacatgaag |
| | S V L Q D N S S K I C Q D M K |
| 695 | gaacctgaagacaactctcctaacacatgccaagtaatatctgta |
| | E P E D N S P N T C Q V I S V |
| 740 | attcaagaccatcctttcaaaatgtaccaagatatggctaaacga |
| | I Q D H P F K M Y Q D M A K R |
| 785 | gaagatctggctcctaaaatgtgccaagaagctgctgtacccaaa |
| | E D L A P K M C Q E A A V P K |
| 830 | atccttccttgtccaacatctgaagacacagctgatctggcagga |
| | I L P C P T S E D T A D L A G |
| 875 | tgctctcttcaagcatatccaaaaccagatgtgcctaaaggctat |
| | C S L Q A Y P K P D V P K G Y |
| 920 | attcttgacacagaccaaaatccagcagaaccagaggaatacaat |
| | I L D T D Q N P A E P E E Y N |
| 965 | gaaacagatcaaggaatagctgagacagaaggcctttttcctaaa |
| | E T D Q G I A E T E C L F P K |
| 1010 | atacaagaaatagctgagcctaaagacctttctacaaaaacacac |
| | I Q E I A E P K D L S T K T H |
| 1055 | caagaatcagctgaacctaaataccttcctcataaaacatgtaac |
| | Q E S A E P K Y L P H K T C N |
| 1100 | gaaattattgtgcctaaagccccctctcataaaacaatccaagaa |
| | E I I V P K A P S H K T I Q E |
| 1145 | acacctcattctgaagactattcaattgaaataaaccaagaaact |
| | T P H S E D Y S I E I N Q E T |
| 1190 | cctgggtctgaaaaatattcacctgaaacgtatcaagaaatacct |
| | P G S E K Y S P E T Y Q E I P |
| 1235 | gggcttgaagaatattcacctgaaatataccaagaaacatcccag |
| | G L E E Y S P E T Y Q E T S Q |
| 1280 | cttgaagaatattcacctgaaatataccaagaaacaccggggcct |
| | L E E Y S P E I Y Q E T P G P |
| 1325 | gaagacctctctactgagacatataaaaataaggatgtgcctaaa |
| | E D L S T E T Y K N K D V P K |
| 1370 | gaatgctttccagaaccacaccaagaaacaggtgggccccaaggc |
| | E C F P E P H Q E T G G P Q G |
| 1415 | caggatcctaaagcacaccaggaagatgctaaagatgcttatact |
| | Q D P K A E Q E D A K D A Y T |
| 1460 | tttcctcaagaaatgaaagaaaaacccaaagaagagccaggaata |
| | F P Q E M K E K P K E E P G I |
| 1505 | ccagcaattctgaatgagagtcatccagaaaatgatgtctatagt |
| | P A I L N E S H P E N D V Y S |
| 1550 | tatgttttgttttaacaatgctcaaccataaagttgtggtccaat |
| | Y V L F * |
| 1595 | ggaaaaaaaaaaaaaaaaaaaaaa |

The coding sequence of human EDAG, SEQ ID NO:5 is shown below. This is a fragment of SEQ ID NO:3 and includes 1452 nucleotides; the stop codon is shown in brackets at the end.

| | |
|---|---|
| atggatttgg gaaaggacca atctcatttg aagcaccatc | 50 |

agacacctga

| | |
|---|---|
| ccctcatcaa gaagagaacc attctccaga agtcattgga | 100 |

acctggagtt

| | |
|---|---|
| tgagaaacag agaactactt agaaaaagaa aagctgaagt | 150 |

gcatgaaaag

| | |
|---|---|
| gaaacatcac aatggctatt tggagaacag aaaaaacgca | 200 |

agcagcagag

| | |
|---|---|
| aacaggaaaa ggaaatcgaa gaggcagaaa gagacaacaa | 250 |

aacacagaat

| | |
|---|---|
| tgaaggtgga gcctcagcca cagatagaaa aggaaatagt | 300 |

ggagaaagca

| | |
|---|---|
| ctggcaccta tagagaaaaa aactgagcca cctgggagca | 350 |

taaccaaagt

| | |
|---|---|
| atttccttca gtagcctccc cgcaaaaagt tgtgcctgag | 400 |

gaacactttt

| | |
|---|---|
| ctgaaatatg tcaagaaagt aacatatatc aggagaattt | 450 |

ttctgagtac

| | |
|---|---|
| caagaaatag cagtacaaaa ccattcttct gaaacatgcc | 500 |

aacatgtgtc

| | |
|---|---|
| tgaacctgaa gacctctctc ctaaaatgta ccaagaaata | 550 |

tctgtacttc

| | |
|---|---|
| aagacaattc ttccaaaata tgccaagaca tgaaggaacc | 600 |

tgaagacaac

| | |
|---|---|
| tctcctaaca catgccaagt aatatctgta attcaagacc | 650 |

atcctttcaa

| | |
|---|---|
| aatgtaccaa gatatggcta aacgagaaga tctggctcct | 700 |

aaaatgtgcc

| | |
|---|---|
| aagaagctgc tgtacccaaa atccttcctt gtccaacatc | 750 |

tgaagacaca

| | |
|---|---|
| gctgatctgg caggatgctc tcttcaagca tatccaaaac | 800 |

cagatgtgcc

| | |
|---|---|
| taaaggctat attcttgaca cagaccaaaa tccagcagaa | 850 |

ccagaggaat

| | |
|---|---|
| acaatgaaac agatcaagga atagctgaga cagaaggcct | 900 |

ttttcctaaa

| | |
|---|---|
| atacaagaaa tagctgagcc taaagacctt tctacaaaaa | 950 |

cacaccaaga

| | |
|---|---|
| atcagctgaa cctaaatacc ttcctcataa aacatgtaac | 1000 |

gaaattattg

| | |
|---|---|
| tgcctaaagc cccctctcat aaaacaatcc aagaaacacc | 1050 |

tcattctgaa

| | |
|---|---|
| gactattcaa ttgaaataaa ccaagaaact cctgggtctg | 1100 |

aaaaatattc

| | |
|---|---|
| acctgaaacg tatcaagaaa tacctgggct tgaagaatat | 1150 |

tcacctgaaa

| | |
|---|---|
| tataccaaga aacatcccag cttgaagaat attcacctga | 1200 |

aatataccaa

| | |
|---|---|
| gaaacaccgg ggcctgaaga cctctctact gagacatata | 1250 |

aaaataagga

| | |
|---|---|
| tgtgcctaaa gaatgctttc cagaaccaca ccaagaaaca | 1300 |

ggtgggcccc

| | |
|---|---|
| aaggccagga tcctaaagca caccaggaag atgctaaaga | 1350 |

tgcttatact

| | |
|---|---|
| tttcctcaag aaatgaaaga aaaacccaaa gaagagccag | 1400 |

gaataccagc

| | |
|---|---|
| aattctgaat gagagtcatc cagaaaatga tgtctatagt | 1450 |

tatgttttgt

| | |
|---|---|
| tt``[taa] | 1452 |

The amino acid sequence of human EDAG (SEQ ID NO:4), 484 residues, is shown separately below.

| | |
|---|---|
| MDLGKDQSHL KHHQTPDPHQ EENHSPEVTG TWSLRNRELL | 50 |

RKRKAEVHEK

| | |
|---|---|
| ETSQWLFGEQ KKRKQQRTGK GNRRGRKRQQ NTELKVEPQP | 100 |

QIEKEIVEKA

| | |
|---|---|
| LAPIEKKTEP PGSITKVFPS VASPQKVVPE EHESEICQES | 150 |

NIYQENFSEY

| | |
|---|---|
| QEIAVQNHSS ETCQHVSEPE DLSPKMYQEI SVLQDNSSKT | 200 |

CQDMKEPEDN

| | |
|---|---|
| SPNTCQVISV IQDHPFKMYQ DMAKREDLAP KMCQEAAVPK | 250 |

ILPCPTSEDT

| | |
|---|---|
| ADLAGCSLQA YPKPDVPKGY ILDTDQNPAE PEEYNETDQG | 300 |

TAETEGLFPK

| | |
|---|---|
| IQEIAEPKDL STKTHQESAE PKYLPHKTCN EITVPKAPSH | 350 |

KTIQETPHSE

| | |
|---|---|
| DYSTEINQET PGSEKYSPET YQEIPGLEEY SPEIYQETSQ | 400 |

LEEYSPEIYQ

| | |
|---|---|
| ETPGPEDLST ETYKNKDVPK ECFPEPHQET GGPQGQDPKA | 450 |

HQEDAKDAYT

| | |
|---|---|
| FPQEMKEKPK EEPGIPAILN ESHPENDVYS YVLF | 484 |

EDAG, also specifically expressed in hematopoietic cells, maps to chromosome 9q22, a region containing breakpoints (for translocation) present in several hematopoietic neoplasms.

The invention is also directed to an isolated nucleic acid molecule that hybridizes with any of the above nucleic acid molecules under stringent hybridization conditions. Preferred stringent conditions include incubation in 6×sodium chloride/sodium citrate (SSC) at about 45° C., followed by a wash in about 0.2×SSC at a temperature of about 50° C.

A preferred nucleic acid molecule as above encodes a protein having an amino acid sequence selected from SEQ ID NO:2 and SEQ ID NO:4 or encodes a biologically active fragment, homologue or other functional derivative of the protein. Preferably, the nucleic acid molecule encodes the protein having the sequence SEQ ID NO:4 (EDAG of human origin) or encodes the biologically active fragment, homologue or other functional derivative of SEQ ID NO:4.

The present invention includes an “isolated” Hemogen or EDAG polypeptide having the sequence SEQ ID NO:2 or SEQ ID NO:4. While the present disclosure exemplifies the full length human and murine proteins (and DNA), it is to be understood that homologues of EDAG from other mammalian species and mutants thereof that possess the characteristics disclosed herein are intended within the scope of this invention.

Also included is a “functional derivative” of EDAG which is means an amino acid substitution variant, a “fragment,” or a “chemical derivative” of EDAG, which terms are defined below. A functional derivative retains measurable EDAG activity, preferably that of binding to an anti-EDAG antibody, or expression in an hematopoietic cells, preferably progenitors, which permits its utility in accordance with the present invention. “Functional derivatives” encompass “variants” and “fragments” regardless of whether the terms are used in the conjunctive or the alternative herein.

A functional homologue must possess the above biochemical and biological activity. In view of this functional characterization, use of homologous proteins EDAG from other species, including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. The amino acid residues (or nucleotides) at corresponding amino acid positions (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to human or murine EDAG nucleic acid molecules. BLAST protein searches can be performed with the XBLAST program, score=50, wordlength=3 to obtain amino acid sequences homologous to human or murine EDAG protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of the EDAG protein described above is characterized as having (a) functional activity of native EDAG, and (b) sequence similarity to a native EDAG protein (such as SEQ ID NO:2 or SEQ ID NO:4, when determined above, of at least about 30% (at the amino acid level), preferably at least about 50%, more preferably at least about 70%, even more preferably at least about 90%.

It is within the skill in the art to obtain and express such a protein using DNA probes based on the disclosed sequences of EDAG. Then, the protein's biochemical and biological activity can be tested readily using art-recognized methods such as those described herein.

Also provided is an expression vector comprising any of the above nucleic acid molecules operatively linked to

(a) a promoter and

(b) optionally, additional regulatory sequences that regulate expression of the nucleic acid in a eukaryotic cell.

The above expression vector may be a plasmid or a viral vector. These vectors include self replicating RNA replicons (DNA-launched or RNA), suicide RNA vectors DNA viruses (such as adenovirus, vaccinia virus, etc.) and RNA virions grown on packaging cell lines.

The vector DNA or RNA may be complexed to gold particles for gene gun-mediated introduction to a host or complexed with other polymers, for example, in controlled release formulations, that enhance delivery to the desired target cells and tissues.

This invention includes a cell transformed or transfected with any of the above nucleic acid molecules or expression vectors. The cell is preferably a eukaryotic cell, more preferably a mammalian cell, most preferably a human cell. The cell may be a hematopoietic cell, preferably a progenitor cell. In another embodiment, the cell is a tumor cell.

A preferred embodiment is an isolated mammalian tumor cell transfected with an exogenous nucleic acid molecule encoding a mammalian EDAG (preferably SEQ ID NO:2 or SEQ ID NO:4) or a biologically active fragment, homologue or other functional derivative thereof, wherein the EDAG is expressed in the cells.

Also provided is a EDAG fusion polypeptide having a first fusion partner comprising all or a—part of a EDAG protein fused

(i) directly to a second polypeptide or,

(ii) optionally, fused to a linker peptide sequence that is fused to the second polypeptide.

The above A EDAG fusion protein may also be fused to a second polypeptide, preferably one or more domains of an Ig heavy chain constant region, preferably having an amino acid sequence corresponding to the hinge, C_{H}2 and C_{H}3 regions of a human immunoglobulin Cyl chain.

The present invention includes antibodies specific to Hemogen and to EDAG, produced by conventional methods. These include polyclonal antisera and monoclonal antibodies. Also included are antigen-binding fragments of these antibodies. The antibodies may be used to detect the presence or measure the amount of Hemogen or EDAG protein in a tissue sample or biological fluid in any conventional immunoassay. The antibodies may therefore be used in diagnostic assays for abnormalities associated with altered expression of these proteins in humans, mice or in the homologues of these proteins in other species.

Thus, in one embodiment is included an antibody that is specific for an epitope of a EDAG protein. The epitope may be a linear or conformational epitope of a polypeptide of SEQ ID NO:2 or SEQ ID NO:4. The antibody is preferably a monoclonal antibody, more preferably a human or humanized (via engineering) monoclonal antibody.

Also provided is a method of using the above antibody to identify or quantitate cells expressing a EDAG polypeptide in a cell population, comprising

(a) contacting cells of the population with the above antibody so that the antibody binds to cells expressing the epitope;

(b) assessing the presence of or quantitating the number of cells to which the antibody is bound.

Another method is provided for isolating cells expressing a EDAG polypeptide from a cell population, comprising

(a) contacting the population with the above antibody so that the antibody binds to cells expressing the epitope;

(b) positively selecting cells to which the antibody has bound or negatively selecting cells to which the antibody has not bound.

Also provided is a method of detecting the presence or quantitating a EDAG polypeptide, fragment or homologue in a sample, comprising the steps of:

(a) contacting the sample with the above antibody such that the antibody binds to any polypeptides or fragments bearing the epitope;

(b) detecting the presence of, or quantitating the polypeptides or fragments bound to the antibody.

The nucleic acid encoding EDAG and the EDAG protein are used in diagnostic methods and kits for evaluating abnormalities in early hematopoiesis such as those that lead to cancer. These molecules are also useful in drug screening for potential agents that stimulate or inhibit early hematopoietic differentiation and may contribute to the inhibition of leukemogenesis or lymphomagenesis. Finally, the protein, biologically active fragments thereof, or antibodies to the protein, are useful as therapeutic agents to treat diseases associated with abnormal early hematopoietic differentiation such as certain forms of leukemia.

### EXAMPLE I

### Experimental Procedures

Cloning and Sequencing of the Mouse Hemogen and the Human EDAG

We have used PCR-based cDNA subtraction to identify differentially regulated genes during mouse cardiac development. Heart tissue, which contains some blood cells, was dissected from mouse embryos at E10.5 and E16.5, and the RNA was extracted. With E10.5 RNA as the tester and E16.5 RNA as the driver, subtraction hybridization was performed using the PCR-Select cDNA subtraction kit (Clontech). The subtracted clones were then sequenced in an automated DNA sequencer. The sequences were searched against GenBank. One clone, 6B2, shared homology with several mouse expressed sequence tags (ESTs). There was no match in the non-redundant database. Three EST clones (accession # AA051237, AI121196 and AI006512) were purchased from Research Genetics and sequenced in both directions to obtain the full-length cDNA, now designated Hemogen, as shown in FIG. 1. The opening reading frame (ORF) was deduced. Additional cDNA clones were also identified through screening the E10 heart cDNA library (Stratagene).

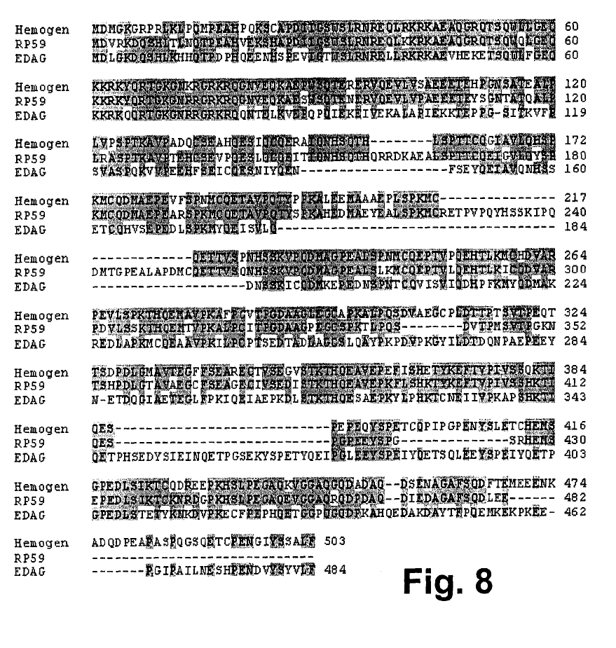

Hemogen also shared homology with several human ESTs. Two human EST clones (accession # T52254 and AA393302) were sequenced and the ORF was deduced. During the process of our study, three human homologous sequences, including two draft human genomic sequences (accession # AC015928 and AL354726) and a human cDNA with hypothetical protein EDAG-1 (accession # AF228713), were deposited to GenBank. The putative ORF that we defined and named “EDAG” shows an additional 175 amino acids at the N-terminus of the previous deposited EDAG-1 sequence. Recently, a rat homologue, RP59 (GenBank # AJ302650) was deposited. The amino acid sequences of Hemogen, EDAG and RP59 were aligned (FIG. 8) using ClustalW program (http://www2.ebi.ac.uk/clustalw/).

The Hemogen and EDAG sequences have been deposited in GenBank and assigned accession numbers AF269248 and AF322875 respectively.

Plasmid Constructs

Mammalian expression vector pcDNA3.1 (Invitrogen) was modified by inserting a FLAG tag into the multiple cloning sites to express the FLAG-fusion protein. This plasmid was re-named pcDNA3.1-FLAG. To generate a mammalian expression vector of Hemogen, the ORF (191-1699 bp) was cloned into pcDNA 3.1-FLAG. The resulting construct, named pcDNA3.1-Hemogen, was sequenced to confirm that the Hemogen ORF was fused in-frame with the FLAG tag at the C-terminus.

Immunostaining

COS-7 cells were cultured in Dulbecco's modified Eagle medium with 10% fetal bovine serum (Gibco BRL). Using Lipofectamine Plus™ reagent (Gibco BRL), 1 μg plasmid DNA of pcDNA3.1-Hemogen was transfected into COS-7 cells to express Hemogen-FLAG fusion protein. In parallel, the same amount of pcDNA3.1-FLAG vector was transfected as a negative control. For immunostaining, 24 hours after transfection cells were fixed in 4% paraformaldehyde for 4 minutes. The endogenous peroxidase was quenched with 0.3% H₂O₂ in methanol. After the treatment with 2% blocking serum, the cells were incubated with the mouse anti-FLAG M2 monoclonal antibody (Sigma), and then, after washing, with the anti-mouse IgG antibody conjugated with peroxidase (Vector). The signals were detected through the reaction of peroxidase with the substrate DAB (Roche).

In vitro Transcription

RNAs synthesized by in vitro transcription were used as the riboprobes for Northern and in situ hybridization. A 224 bp EcoRI-ApaI fragment of Hemogen cDNA was cloned into pBluescript-SKII vector to produce riboprobe. The conditions for in vitro transcription were the following: 1 μg linearized DNA, lx transcription buffer (Roche), 10 mM DTT, 2 μl DIG RNA labeling mix (Roche), 20 units RNase inhibitor, 10 units RNA polymerase in 20 μl volume. The reaction was incubated at 37° C. for 2 hours and then digested with 2 units RNase-free DNaseI at 37° C. for 15 minutes to remove the template DNA.

In situ Hybridization

Mouse embryos and tissues were fixed in 4% paraformaldehyde overnight at 4° C., embedded in paraffin and sectioned. In situ hybridization was carried out using a modification of a previously described method (Wilkinson, 1992). Tissue sections were pretreated with 0.2N HCl for 20 minutes, 10 μg/ml proteinase K for 15 minutes at 37° C., 4% formaldehyde for 20 minutes, and 0.5% acetic anhydride in 0.1M TEA (pH 8.0) for 10 minutes. The prehybridization was performed in the hybridization buffer (50% formamide, 5×SSC, pH 4.5, 2% blocking reagent (Roche), 0.1% Tween 20, 0.5% CHAPS, 50 μg/ml yeast RNA, 5 mM EDTA, 50 μg/ml heparin) at 60-65° C. for 1 hour. The hybridization was done with 1 μg/ml digoxigenin-labeled RNA probes at 60-65° C. overnight. Non-specific reactants were removed by three 15-minute washes in 50% formamide, 2×SSC and 0.1% CHAPS at 60-65° C. The samples were incubated with alkaline phosphatase-conjugated anti-digoxigenin antibody (Roche) overnight at 4° C., and then washed to remove the non-specific binding. Signals were developed with the substrate NBT/BCIP (Roche).

RNA Isolation and Northern Analysis

RNAs were extracted from tissues or cells with TRIzol reagent (Gibco BRL), and fractionated in 1.2% agarose-formaldehyde gel. The Northern blots were performed by standard methods (Sambrook et al., 1989). Human tissue blot was purchased from Clontech.

Reverse Transcription Polymerase Chain Reaction (RT-PCR)

1-5 μg total RNA was used to synthesize first-strand cDNA using SuperScript™ reverse transcriptase (Gibco BRL) in 20 μl reaction. The PCR reaction was performed with Taq DNA polymerase (Gibco BRL) for 30 cycles.

Primer pairs were:

| | |
|---|---|
| Hemogen: | |
| 5′-AAACACACCTCTCTCCTACCAC-3′ and | (SEQ ID NO:6) |
| 5′-CCTACTTTCTGGGCTCCTTCTG-3′. | (SEQ ID NO:7) |
| EDAG: | |
| 5′-AAGCACCATCAGACACCTGACC-3′ and | (SEQ ID NO:8) |
| 5′-TGCTTGAAGAGAGCATCCTGCC-3′. | (SEQ ID NO:9) |
| Histone H3: | |
| 5′-CCACTGAACTTCTGATTCGC-3′ and | (SEQ ID NO:10 |
| 5′-GGGTGCTAGCTGGATGTCTT-3′. | (SEQ ID NO:11) |

The PCR products of Hemogen, EDAG and Histone are 881 bp, 751 bp and 214 bp respectively.

Expression Analysis of Hemogen and EDAG by RT-PCR

Total RNA was extracted from a variety of freshly isolated tissues, flow-sorted BM cells, cultured cells and transformed cell lines. Gene expression was analyzed by RT-PCR. The cells were isolated as previously described (Nicholson et al., 2000). In brief, adult mouse BM cells were sorted using different mAbs in a fluorescence activated cell sorter (FACS Vantage, Becton Dickinson). Natural killer cells were generated as previously described (Hirayama et al., 1998) by culturing mouse newborn liver cells for 21 days with 500 units/ml recombinant human IL-2. BM-derived macrophages were obtained as previously described (Li and Chen, 1995). All human tissues were obtained with approval from the Institutional Review Board of Wayne State University.

Adult BM cells were isolated from fragments of ribs that were removed from patients undergoing thoracic surgery. Young thymus tissue was obtained from children undergoing cardiac surgery. CD34+cells and monocytes were obtained from the blood of cancer patients undergoing peripheral mobilization for autologous transplant. T cells were obtained from blood, cultured with IL-2 for several weeks and were >99% CD3+. Hematopoietic cell lines K562, U937 and non-hematopoietic cell lines 24SV48, HUVEC and SKBR3 were cultured in RPMI-1640 medium with 10% calf serum.

### EXAMPLE II

### Cloning and Sequence Analysis of a Novel Murine Gene Hemogen

We initially performed a PCR-based cDNA subtraction, aiming to identify developmentally regulated genes in mouse E10.5 and E16.5 heart tissues. A number of differentially expressed genes were identified in this screening. Possibly because blood cells were trapped in heart tissue, some hematopoietic genes, such as embryonic ε and βH1 globins, were also cloned.

One of the clones, 6B2, showed differential expression at E10.5. Based on a search in GenBank, this clone showed sequence similarity and homology with several mouse ESTs, e.g., GenBank accession numbers AA051237, AI121196 and AI006512. These three independent clones were sequenced to obtain the full-length cDNA sequences from which the amino acid sequence was deduced (FIG. 1). This novel gene, now designated Hemogen (hemopoietic gene), encodes 503 amino acids with a calculated molecular weight 55,043 Da and a pI of 4.84.

Sequence analysis showed the first ATG is the translation initiation codon and the surrounding sequence AAGATGG is consistent with the Kozak consensus sequence (purine at position −3 and G at position +4 (Kozak, 1997). Stop codons exist in all three reading frames upstream of the presumed initiation codon. The polyadenylation signal sequence is ATTAAA (2288-2293 nt), a most common variant of the canonical sequence AATAAA (Graber et al., 1999).

The putative Hemogen protein has a basic N-terminal domain (34-78 residues with a net charge of +15) and an acidic C-terminal domain (450-480 residues with a net charge −11). In the basic domain, the region from residues 34-50 is predicted (at a window size of 14) to be a coiled-coil domain (Lupas, 1996), which is implicated in protein polymerization. Residues 61-78 contain a bipartite nuclear localization signal suggesting that Hemogen is a nuclear protein (Dingwall and Laskey, 1991). Analysis of amino acid composition shows that usages of proline (10.3%), glutamate (8.7%) and glutamic acid (11.9%) are higher than average (Brendel et al., 1992).

### EXAMPLE III

Hemogen Encodes a Nuclear Protein

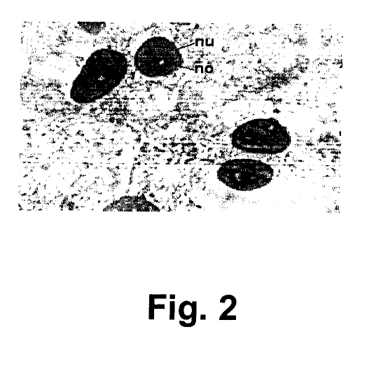

To confirm that the nuclear localization signal in Hemogen protein was functional, we transfected the mammalian expression vector containing the Hemogen-FLAG fusion gene into COS-7 cells, and used anti-FLAG antibody to determine the subcellular localization of this protein. Hemogen was found in cell nuclei but not in nucleoli or cytoplasm (FIG. 2).

### EXAMPLE IV

### Sites of Hemogen Expression Shift With Sites of Hematopoiesis During Ontogeny

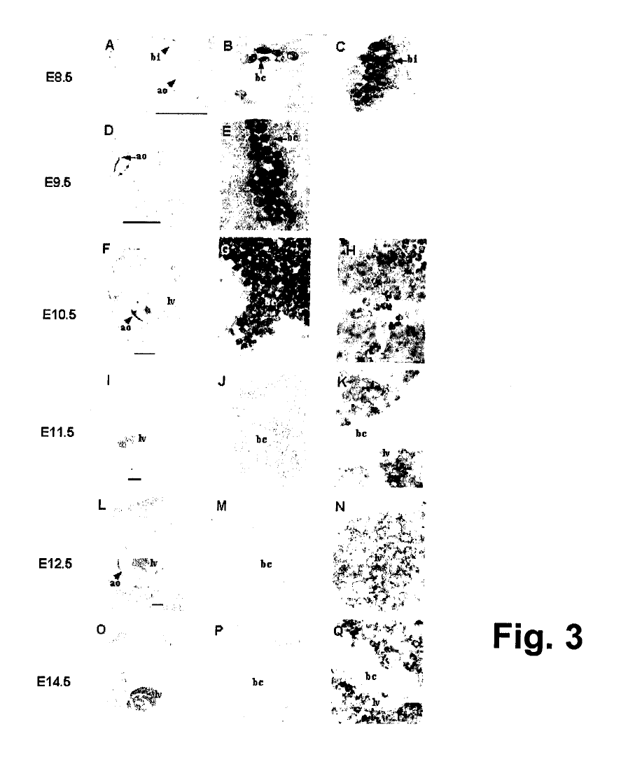

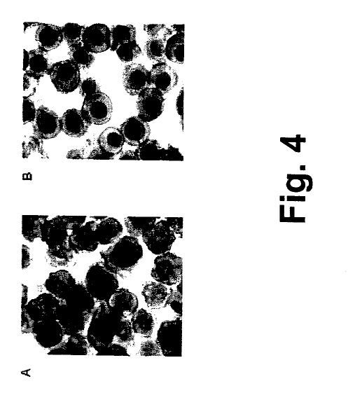

To study the expression pattern of Hemogen during mouse development, we used in situ hybridization and Northern blotting to detect mRNA transcripts. Hemogen was expressed during early embryogenesis at E8.5 in the blood islands of the yolk sac and in the circulating primitive blood cells (FIG. 3A,B,C). The blood islands are the first sites to produce primitive blood cells. Expression in circulating blood cells was highly detectable at E9.5 and E10.5 (FIG. 3D,E,F,G). As liver organogenesis emerged at E10.5, Hemogen expression became detectable in the developing hepatic primordia (FIG. 3F,H). Fetal liver is the primary site of definitive blood cells generation during fetal stages. From E11.5, Hemogen was exclusively expressed in the fetal liver (FIG. 3I, K), while expression in circulating blood cells was downregulated dramatically to undetectable levels (FIG. 3J). The same expression patterns were observed in E12.5 and E14.5 embryos (FIG. 3L,M,N,O,P,Q). Primitive erythrocytes at E10.5 and E12.5 are easily distinguished morphologically by hematoxylin staining. The primitive erythrocytes at E10.5 showed higher nucleus/cytoplasm ratios than those at El 2.5 (FIG. 4A,B).

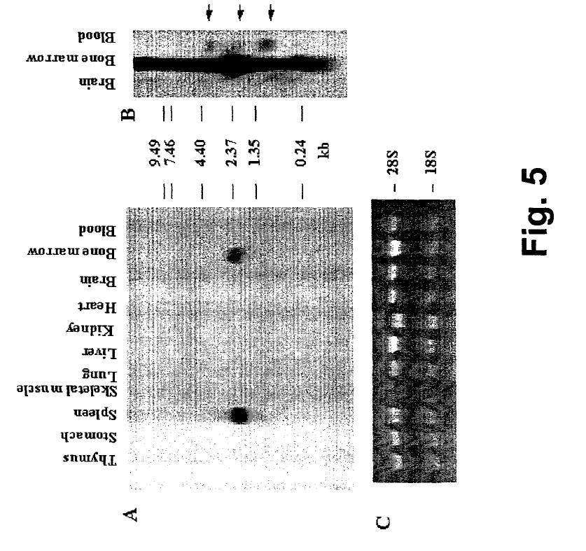

We examined the tissue distribution of Hemogen in adult mice by Northern blots. A 2.4 kb transcript was specifically expressed in the BM and spleen (FIG. 5A). When the same filter was overexposed, a weak signal was detected in the peripheral blood, and two additional transcripts at 1.1 and 3.7 kb were also identified (1.1 kb band giving the strongest signal. (FIG. 5B). This suggested multiple isoforms of Hemogen in peripheral blood cells. No expression was detected in the thymus and various non-hematopoietic tissues including brain, heart, kidney, liver, lung, skeletal muscle and stomach.

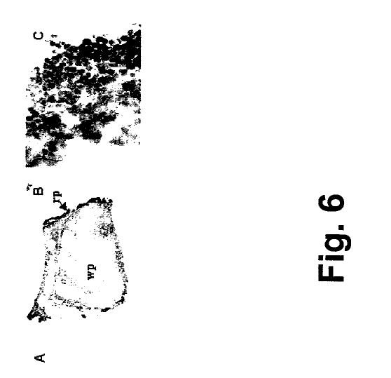

In adult spleen, the red pulp is active in erythropoiesis (while the white pulp is a lymphoid tissue that contains mature B and T lymphocytes (van Ewijk and Nieuwenhuis, 1985)). To determine the localization of Hemogen expression in adult spleen, we performed in situ hybridization. As shown in FIG. 6B, Hemogen was expressed in the red pulp but not in the white pulp. Higher magnification (FIG. 6C) revealed that the positively stained cells were presumably erythroid precursor cells. Hemogen was detected in about 70% of the cells in the red pulp.

These results demonstrate that Hemogen is expressed in both primitive and definitive hematopoiesis. It is sequentially expressed in the active hematopoietic sites, such as yolk sac, fetal liver, BM and spleen. Hemogen expression is highly specific to the hematopoietic system throughout development since no transcripts were detected in any non-hematopoietic tissues.

### EXAMPLE V

### Hemoen is Primarily Expressed in Immature Hematopoietic Cells

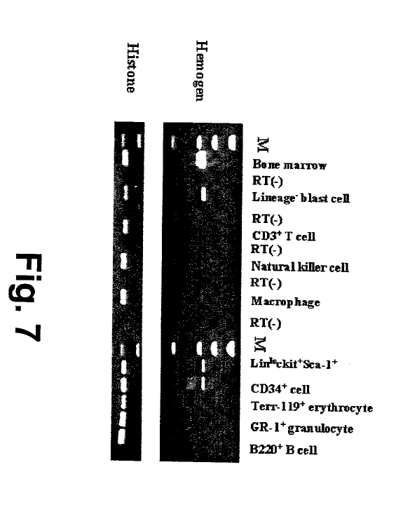

To further investigate which hematopoietic cells express Hemogen, we purified adult mouse BM cells by flow cytometry using monoclonal antibodies and assessed Hemogen expression by RT-PCR. As shown in FIG. 7, Hemogen was primarily expressed in Lineage⁻blast cells, LinₗₒcKit⁺Sca-1⁺pluripotent stem cells and CD34⁺ stem cells. Previous studies have shown that these three cell types (Spangrude et al., 1988, Li and Johnson, 1995, Krause et al., 1996) are enriched of early multipotential stem cells. Low levels of expression were found in cultured macrophages and natural killer cells. However no expression was detected in freshly isolated CD3⁺ T cells, B220⁺ B cells, Terr-119⁺ erythrocytes and GR-1⁺ granulocytes, which are all differentiated blood cells. Hence, Hemogen is differentially expressed in the hematopoietic progenitor cells and downregulated in differentiated blood cells. This notion is consistent with the observation that Hemogen is expressed in the active hematopoietic sites known to harbor hematopoietic progenitor cells (FIGS. 3,5), whereas expression is diminished in the peripheral blood that contains mainly mature blood cells (FIG. 5).

### EXAMPLE VI

### A Human Homologous Gene of Hemogen, EDAG, Maps to Chromosome 9q22, a Location Associated With Blood Disease Breakpoints

To search for human homologues of Hemogen, we identified two human ESTs (accession # T52254 and AA393302) in GenBank. At the time the present study was underway, three human homologous sequences, including a hypothetical protein EDAG-1 (accession #AF228713) and two draft human genomic sequences (accession # AC015928 and AL354726) were deposited to GenBank. According to the GenBank entry, EDAG-1 encodes a 309 amino acid protein with previously unknown function. A BLAST search (Altschul et al., 1997) showed that residues 216-503 Hemogen protein shared 42% identity and 55% similarity with EDAG-1 protein. By sequence analysis, the two draft genomic sequences AC015928 and AL354726 were found to contain the EDAG-1 gene.

Moreover, from the cDNA sequences of human EST clones T52254 and AA393302, we concluded that this cDNA is an isoform of EDAG-1 cDNA. Another upstream ATG translation initiation codon defined a longer ORF encoding a 484 amino acid polypeptide (FIG. 8), This was designated EDAG to distinguish it from “EDAG-1” that lacks the N-terminal 175 amino acids. The same ORF of 484 amino acids was also deduced from the genomic sequence.

Protein sequence alignment showed overall 43% identity between Hemogen and EDAG (FIG. 8). However, the nuclear localization signal and coiled-coil domain are highly conserved with 94% and 76% similarity respectively, suggesting that EDAG may also be a nuclear protein. When we were preparing this manuscript, a rat homologue, RP59 (accession # AJ302650) was deposited in GenBank. Hemogen has 70% identity with RP59. The nuclear localization signal and coiled-coil domain are almost identical (FIG. 8).

We searched the human gene-mapping databases to determine the chromosome localization of EDAG. By searching the Map Viewer at NCBI (http://www.ncbi.nlm.nih.gov/genome/guide/), the genomic sequence AC015928 was located in the region 85.1-85.3 Mb of chromosome 9 in the GenBank map. This ˜160 kb BAC clone was also found to contain the forkhead box E1 (FOXE1/FKHL15) gene that has been mapped to chromosome 9q22 (Chadwick et al., 1997). Therefore, the genomic clone AC015928, containing FOXE1/FKHL15 and EDAG, is located at the same position-9q22. Furthermore, by electronic PCR at NCBI (http://www.ncbi.nlm.nih.gov/genome/sts/epcr.cgi), EDAG was found to contain the STS marker SHGC-33415. A search of GeneMap'99 (http://www.ncbi.nlm.nih.gov/genemap/) and GDB database (http://gdbwww.gdb.org/gdb/advancedSearch.html) revealed that the marker SHGC-33415 is on the long arm of chromosome 9 between the markers D9S287 and D9S176, which correspond to the 9q22 region on the cytogenetic ideogram. Therefore the human gene EDAG, homologous with Hemogen, maps to chromosome 9q22, which correlates with breakpoints detected in several human hematological neoplasms, such as acute myeloid leukemia (Mitelman, 1991; Mitelman et al., 1997).

### EXAMPLE VII

### EDAG is Also Specifically Expressed in Human Hematopoietic Tissues and Cells

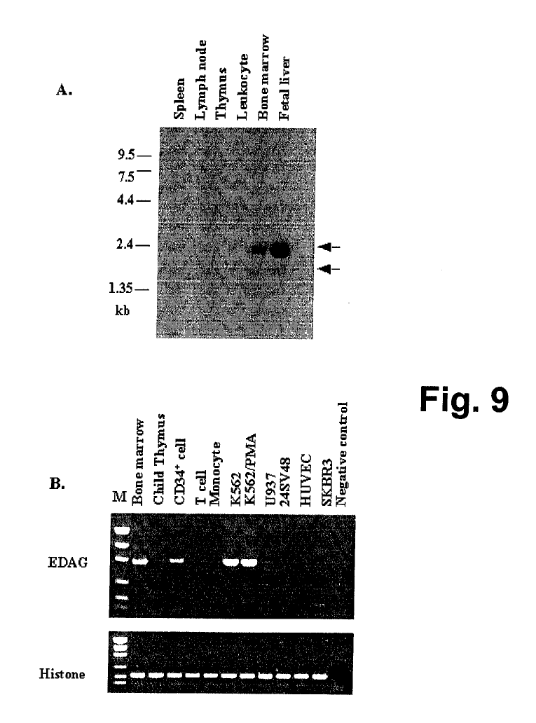

The hybridization of an EDAG cDNA probe with human tissue RNA blots revealed that EDAG was expressed in the active hematopoietic organs, BM and fetal liver (FIG. 9A). Two isoforms, a 2.4 kb major isoform and a 1.8 kb minor isoform, were detected. No expression was found in the spleen, lymph node, thymus or peripheral blood leukocytes (FIG. 9A). It is noteworthy that in human adults, the spleen and thymus are inactive in hematopoiesis under normal condition. RT-PCR permitted detection of high level of transcripts in the myelogenous leukemia cell line K562, K562 stimulated with phorbol myristate acetate (PMA), adult BM and CD34⁺ progenitor cells. Low level of expression appeared in thymus of a child and in cells of the histiocytic lymphoma (macrophage-like) cell line U-937. No expression was detected in cultured blood T cells, monocytes or in other non-hematopoietic cell lines including SV-40 transformed thymus epithelial cell line 24SV48, endothelial cell line HUVEC and breast epithelial cell line SKBR3 (FIG. 9B). Thus, EDAG is specifically expressed in hematopoietic cells, and expression is developmentally regulated.

### Discussion

Described herein are a novel murine gene Hemogen and its human homologue EDAG that are specifically expressed in hematopoietic tissues. Hemogen exhibited highly specific expression in hematopoiesis throughout development. During primitive hematopoiesis, Hemogen is detectable in blood islands and in circulating primitive erythrocytes. During definitive hematopoiesis, Hemogen is expressed in the fetal liver as early as the time of formation of hepatic primordia at E10.5. After El 1.5, expression was limited to the fetal liver. In adult mice, Hemogen was expressed in BM, spleen and very weakly in peripheral blood but not in other tissues. Very few genes exhibit such tissue- and stage-specific expression patterns in the blood system throughout all developmental stages. Given its presence as a nuclear factor, Hemogen is expected to play a regulatory roles in hematopoiesis.

The expression of Hemogen in primitive erythroid cells is of particular interest. During mouse embryogenesis there are two distinct populations of erythrocytes, the primitive (nucleated, yolk sac derived) and definitive (enucleated, fetal liver derived) erythrocytes. Before E11, all erythrocytes in the blood stream are primitive. After E11, the fetal liver begins to generate definitive erythrocytes that gradually replace primitive erythrocytes in the circulation. Primitive erythrocytes usually produce embryonic globins, but begin to synthesize adult globins after E11 (Brotherton et al., 1979). The nuclei of primitive erythrocytes gradually condense during embryonic development. By in situ hybridization, expression of Hemogen in primitive erythrocytes was abundant before E11.5 but was dramatically downregulated after E11.5. This downregulation correlates with primitive erythroid differentiation in terms of morphological and molecular changes. Similar downregulation has been observed in transcription factors EKLF (Southwood et al., 1996) and SCL/tal-1 (Elefanty et al., 1999) that are crucial for erythroid development (Robb et al., 1995; Shivdasani et al., 1995; Nuez et al., 1995; Perkins et al., 1995).

Evidently differential gene expression is important in controlling developmental events in hematopoiesis. A variety of genes are down- or up-regulated during cell differentiation. For examples, the transcription factors SCL/tal-1, GATA-2 and GATA-1 expression extinguishes during erythroid differentiation from primitive (CD34⁺, CD38⁻) progenitors (Cheng et al., 1996). Like these factors, Hemogen was differentially expressed in immature progenitor cells and downregulated in mature blood cells. The results indicate a role for Hemogen/EDAG in blood cell differentiation.

EDAG is the human homologue of Hemogen, and similarly, EDAG is closely tied in with hematopoiesis. Based on sequences in GenBank (accession # AF228713), EDAG cDNA was cloned from the fetal liver, an active site of hematopoiesis. Moreover, EDAG was specifically expressed in a variety of hematopoietic cells and tissues but not in non-hematopoietic cells.

By data mining, we mapped EDAG to chromosome 9q22. This locus is of particular interest since a number of breakpoints associated with human blood diseases have been mapped to this region (see the website http://www.ncbi.nlm.nih.gov/CCAP/mitelsum.cgi), for examples, acute myeloid leukemia with deletions del(9)(q22), del(9)(q12q22), del(9)(q13q22) or translocation t(9;10)(q22; q22) (Kao et al., 1986; Mitelman, 1991; Mitelman et al., 1997; Sreekantaiah et al., 1989; Yunis et al., 1984). A genetic disease, familial hemophagocytic lymphohistiocytosis (HPLH1), also maps to 9q21.3-q22 (Ohadi et al., 1999).

It was suggested that the chromosome region 9q21-q22 contains a cluster of leukemia breakpoints, and genes important for leukemogenesis appear to reside in this region (Sreekantaiah et al., 1989). Now in light of the evidence presented here that EDAG is involved in hematopoiesis, EDAG appears to be a candidate gene for involvement in these diseases.

In summary, we cloned Hemogen, a novel murine gene expressed in cells and tissues that coincide with active hematopoiesis. We have also discovered its human homologue that is specifically expressed in hematopoietic tissues and maps to leukemia breakpoints of a human chromosome.

### Documents Cited

Altschul, S. F., Madden, T. L., Schaffer, A. A., Zhang, J., Zhang, Z., Miller, W. and Lipman, D. J., 1997. Gapped BLAST and PSI-BLAST: a new generation of protein database search programs. Nucleic Acids Res 25, 3389-3402.

Brendel, V., Bucher, P., Nourbakhsh, I. R., Blaisdell, B. E. and Karlin, S., 1992. Methods and algorithms for statistical analysis of protein sequences. Proc Natl Acad Sci U S A 89, 2002-2006.

Brotherton, T. W., Chui, D. H., Gauldie, J. and Patterson, M., 1979. Hemoglobin ontogeny during normal mouse fetal development. Proc Natl Acad Sci U S A 76, 2853-2857.

Chadwick, B. P., Obermayr, F. and Frischauf, A. M., 1997. FKHL15, a new human member of the forkhead gene family located on chromosome 9q22. Genomics 41, 390-396.

Cheng, T., Shen, H., Giokas, D., Gere, J., Tenen, D. G. and Scadden, D. T., 1996. Temporal mapping of gene expression levels during the differentiation of individual primary hematopoietic cells. Proc Natl Acad Sci U S A 93, 13158-13163.

Dingwall, C. and Laskey, R. A., 1991. Nuclear targeting sequences—a consensus? Trends Biochem Sci 16, 478-481.

Dzierzak, E. and Medvinsky, A., 1995. Mouse embryonic hematopoiesis. Trends Genet 11, 359-366.

Elefanty, A. G., Begley, C. G., Hartley, L., Papaevangeliou, B. and Robb, L., 1999. SCL expression in the mouse embryo detected with a targeted lacZ reporter gene demonstrates its localization to hematopoietic, vascular, and neural tissues. Blood 94, 3754-3763.

Engel, I. and Murre, C., 1999. Transcription factors in hematopoiesis. Curr Opin Genet Dev 9, 575-579.

Graber, J. H., Cantor, C. R., Mohr, S. C. and Smith, T. F., 1999. In silico detection of control signals: mRNA 3′-end-processing sequences in diverse species. Proc Natl Acad Sci U S A 96, 14055-14060.

Hirayama, M., Genyea, C., Brownell, A. and Kaplan, J., 1998. IL-2-activated murine newborn liver NK cells enhance engraftment of hematopoietic stem cells in MHC-mismatched recipients. Bone Marrow Transplant 21, 1245-1252.

Kao, Y. S., Sartin, B. W., Van Brunt, J. and Hew, A. Y., Jr., 1986. Interstitial 9q deletion (q12q22) in two cases of acute myeloblastic leukemia. Cancer Genet Cytogenet 19, 365-366.

Kozak, M., 1997. Recognition of AUG and alternative initiator codons is augmented by G in position +4 but is not generally affected by the nucleotides in positions +5 and +6. EMBO J 16, 2482-2492.

Krause, D. S., Fackler, M. J., Civin, C. I. and May, W. S., 1996. CD34: structure, biology, and clinical utility. Blood 87, 1-13.

Li, C. L. and Johnson, G. R., 1995. Murine hematopoietic stem and progenitor cells: I. Enrichment and biologic characterization. Blood 85, 1472-1479.

Li, Y. and Chen, B., 1995. Differential regulation of fyn-associated protein tyrosine kinase activity by macrophage colony-stimulating factor (M-CSF) and granulocyte-macrophage colony-stimulating factor (GM-CSF). J Leukoc Biol 57, 484-490.

Lupas, A., 1996. Prediction and analysis of coiled-coil structures. Methods Enzymol 266, 513-525.

Mitelman, F., 1991. Catalog of chromosome aberrations in cancer. Wiley-Liss, New York.

Mitelman, F., Mertens, F. and Johansson, B., 1997. A breakpoint map of recurrent chromosomal rearrangements in human neoplasia [see comments]. Nat Genet 15 Spec No, 417-474.

Nicholson, R. H., Pantano, S., Eliason, J. F., Galy, A., Weiler, S., Kaplan, J., Hughes, M. R. and Ko, M. S., 2000. Phemx, a novel mouse gene expressed in hematopoietic cells maps to the imprinted cluster on distal chromosome 7. Genomics 68, 13-21.

Nuez, B., Michalovich, D., Bygrave, A., Ploemacher, R. and Grosveld, F., 1995. Defective haematopoiesis in fetal liver resulting from inactivation of the EKLF gene. Nature 375, 316-318.

Ohadi, M., Lalloz, M. R., Sham, P., Zhao, J., Dearlove, A. M., Shiach, C., Kinsey, S., Rhodes, M. and Layton, D. M., 1999. Localization of a gene for familial hemophagocytic lymphohistiocytosis at chromosome 9q21.3-22 by homozygosity mapping. Am J Hum Genet 64, 165-171.

Orkin, S. H., 1995. Transcription factors and hematopoietic development. J Biol Chem 270, 4955-4958.

Perkins, A. C., Sharpe, A. H. and Orkin, S. H., 1995. Lethal beta-thalassaemia in mice lacking the erythroid CACCC-transcription factor EKLF. Nature 375, 318-322.

Robb, L., Lyons, I., Li, R., Hartley, L., Kontgen, F., Harvey, R. P., Metcalf, D. and Begley, C. G., 1995. Absence of yolk sac hematopoiesis from mice with a targeted disruption of the scl gene. Proc Natl Acad Sci U S A 92, 7075-7079.

Rowley, J. D., 1998. The critical role of chromosome translocations in human leukemias. Annu Rev Genet 32, 495-519.

Sambrook, J., Fritsch, E. F. and Maniatis, T., 1989. Molecular cloning: a laboratory manual. Cold Spring Harbor Laboratory, Cold Spring Harbor, N.Y.

Sawyers, C. L., 1998. Molecular abnormalities in myeloid leukemias and myelodysplastic syndromes. Leuk Res 22, 1113-1122.

Seifert, M. F. and Marks, S. C., Jr., 1985. The regulation of hemopoiesis in the spleen. Experientia 41, 192-199.

Shivdasani, R. A., Mayer, E. L. and Orkin, S. H., 1995. Absence of blood formation in mice lacking the T-cell leukaemia oncoprotein tal-l/SCL. Nature 373, 432-434.

Sieweke, M. H. and Graf, T., 1998. A transcription factor party during blood cell differentiation. Curr Opin Genet Dev 8, 545-551.

Southwood, C. M., Downs, K. M. and Bieker, J. J., 1996. Erythroid Kruppel-like factor exhibits an early and sequentially localized pattern of expression during mammalian erythroid ontogeny. Dev Dyn 206, 248-259.

Spangrude, G. J., Heimfeld, S. and Weissman, I. L., 1988. Purification and characterization of mouse hematopoietic stem cells. Science 241, 58-62.

Sreekantaiah, C., Baer, M. R., Preisler, H. D. and Sandberg, A. A., 1989. Involvement of bands 9q21-q22 in five cases of acute nonlymphocytic leukemia. Cancer Genet Cytogenet 39, 55-64.

van Ewijk, W. and Nieuwenhuis, P., 1985. Compartments, domains and migration pathways of lymphoid cells in the splenic pulp. Experientia 41, 199-208.

Wilkinson, D. G., 1992. In situ hybridization: a practical approach. IRL Press at Oxford University Press, Oxford; New York, The Practical approach series.

Yunis, J. J., Brunning, R. D., Howe, R. B. and Lobell, M., 1984. High-resolution chromosomes as an independent prognostic indicator in adult acute nonlymphocytic leukemia. N Engl J Med 311, 812-818.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. An isolated nucleic acid molecule that encodes a mammalian protein Hemogen/EDAG that is selectively expressed in developing or immature hematopoietic cells.

2. The nucleic acid molecule of claim 1 that comprises a nucleotide sequence selected from SEQ ID NO:1 or SEQ ID NO:5.

3. An isolated nucleic acid molecule that hybridizes with the nucleic acid molecule of claim 1 under stringent hybridization conditions.

4. An isolated nucleic acid molecule that hybridizes with the nucleic acid molecule of claim 2 under stringent hybridization conditions.

5. The nucleic acid molecule of claim 2 that comprises the nucleotide sequence SEQ ID NO:5.

6. The nucleic acid molecule of claim 1 that encodes a protein having an amino acid sequence selected from SEQ ID NO:2 and SEQ ID NO:4 or encodes a biologically active fragment, homologue or other functional derivative of said protein.

7. The nucleic acid molecule of claim 6 that encodes said protein having the sequence SEQ ID NO:4 or encodes said biologically active fragment, homologue or other functional derivative of SEQ ID NO:4.

8. An expression vector comprising the nucleic acid of claim 1 operatively linked to
(a) a promoter and
(b) optionally, additional regulatory sequences that regulate expression of said nucleic acid in a eukaryotic cell.

9. An expression vector comprising the nucleic acid of any of claims 2-7, operatively linked to
(a) a promoter and
(b) optionally, additional regulatory sequences that regulate expression of said nucleic acid in a eukaryotic cell.

10. A cell transformed or transfected with the vector of claim 8.

11. A cell transformed or transfected with the vector of claim 9.

12. A polypeptide that is selectively expressed in developing or immature hematopoietic cells, encoded by a nucleic acid molecule having the sequence SEQ ID NO:1 or SEQ ID NO:5, or a fragment, homologue or functional derivative of said polypeptide.

13. The polypeptide of claim 12 having the amino acid sequence SEQ ID NO:2 or SEQ ID NO:4, or a fragment, homologue or equivalent of said polypeptide.

14. An antibody that is specific for an epitope of the polypeptide of claim 12.

15. An antibody that is specific for an epitope of the polypeptide of claim 13.

16. The antibody of claim 14 or 15 that is a monoclonal antibody.

17. A method of identifying or quantitating cells expressing a EDAG polypeptide on in a cell or tissue sample, comprising
(a) contacting the sample with the antibody of claim 16, so that said antibody binds to cells expressing said epitope;
(b) assessing the presence of, or quantitating the number of, cells to which said antibody is bound.

18. A method of detecting the presence or quantitating a EDAG polypeptide, fragment or homologue in a sample, comprising the steps of:
(a) contacting the sample with the antibody of claim 16 such that the antibody binds to any polypeptides or fragments bearing said epitope;
(b) detecting the presence of, or quantitating the polypeptides or fragments bound to said antibody.

19. A method for detecting an abnormality in early hematopoiesis associated with an abnormal amount of EDAG protein in a biological fluid sample, a cell sample or a tissue sample suspected of said abnormality, comprising:
(a) determining the amount of EDAG in said sample in accordance with claim 18,
(b) comparing the amount determined in step (a) with the amount of EDAG polypeptide in a normal or control sample of said biological fluid, cells or tissue, or with a predetermined normal value;
wherein if the amount of EDAG determined in step is significantly lower or higher than said control or value, said sample is detected as abnormal.


SEQUENCE LISTING (11 sequences)

SEQ ID NO: 1 - DNA, 2331 bp, Mus musculus
  CDS (191)..(1699)
ggaatttgcccacagctgtggtctaaccagataaaacttttaggcgggaagtgacaagcaaacttgaatgtgtgtgtctgtggcttttttttttcttcatcttcatttgaagtgttggtcttgatattttcaaaaagcttttaggctgcctgtgaagtcaaagccaataccaagaaggcatcgtggcaagatggacatggggaagggccgacctcgtctgaagctcccccagatgcctgaagctcacccacagaagtcctgtgctccagacatcattggatcttggagtctgagaaacagagaacaactgaggaagagaaaagctgaggcccaggggaggcagacatcacaatggctccttggagaacagaaaaaacgcaagtatcagagaacaggaaaaggaaataaaagaggccgaaagagacaagggaacgtggagcaaaaggcagagccttggtcacaaacagaaagggaaagggtgcaagaggtattggtatctgctgaggaagaaaccgagcaccctgggaactctgcaactgaagccctccccttggtcccatcccccacaaaagctgtgcctgcagatcagtgttctgaagcacaccaagaaagcattcaatgtcaagaaagagcaatacagaaccattctcaaacacacctctctcctaccacatgccaaggaatagcagtacttcaacattctcctaaaatgtgccaagatatggccgaacctgaggtattctctcctaacatgtgccaggagacagctgtgccccaaacctatcctcccaaagcacttgaagaaatggctgcagccgagccactctctcctaaaatgtgccaggaaacaactgtgtccccaaaccattcttccaaagtgccccaagatatggctggacctgaggctctctctcctaacatgtgccaggaaccaactgtgcctcaagaacatactttgaaaatgtgccatgatgtggccagacctgaagtcctctctcctaaaacacatcaagagatggctgttccaaaagcctttccctgtgtaacacctggagatgctgctggcctggaaggatgcgccccaaaagccctcccccaatcagatgtcgctgaaggctgtccacttgacacaacccccacgtcagtcacaccagaacaaaccacttccgacccagatctgggaatggctgtgactgaaggcttcttttctgaagccagagaatgcactgtttctgaaggcgtttctacaaagacacaccaagaagcagttgaacctgaattcatttctcacgagacttataaagaattcactgtgcctatagtttcttctcagaaaacaatccaagaatcacctgagcctgaacaatattcacctgaaacatgtcaaccaatacctgggcctgagaactattcactggaaacctgccatgaaatgtcggggcctgaagacctctctatcaagacctgtcaggacagggaggagcctaaacacagccttccagaaggagcccagaaagtaggtggggcccaagggcaggacgctgatgcacaggacagcgagaacgctggtgctttctctcaagattttacagaaatggaggaagaaaacaaagcagatcaagatccggaagctccagcaagcccacaaggttctcaagagacctgcccagaaaatggcatctacagctctgctctattttaacagtgctcagtgatggagctgcagtccagctcaatacagcatacatatctcttgtggtttcactgaaacactgcagcaatcactaaaatttgcattgctattttaacttatgcttttttttctatttgtagctcttatctaaaagagagaactaacatttttaaggctctaacacatagacaatagtgtgtgtgtgtgtgtgtgtgtgtgtgtgtgtgtgtgccgtgtgagcacctgagggtgtggatttgtatatgggggaagacagaacaggggaaaggttgagtagttgattttcccctctaagaggaaacatatatttggtagttctgaggagaagatagcaattcaatatgaacacttagtgtttttgaaagtatacagattcttgtaagtcttgtcaactattgatgttgtaacaacatcagaattttattcgagctttacacgtctctgagttgatctgaacaattcttattctaaaagttcttgcaaattattttggaattgataattgtcacttatttctgtgtgaacctgaaccttctatttctattttttaaactgtgtttgtaaaaaatgtacattaaatcattactatggtcttaaaaaaaaaaaaaaaaaaaaaa

SEQ ID NO: 2 - protein, 503 aa, Mus musculus
MDMGKGRPRLKLPQMPEAHPQKSCAPDIIGSWSLRNREQLRKRKAEAQGRQTSQWLLGEQKKRKYQRTGKGNKRGRKRQGNVEQKAEPWSQTERERVQEVLVSAEEETEHPGNSATEALPLVPSPTKAVPADQCSEAHQESIQCQERAIQNHSQTHLSPTTCQGIAVLQHSPKMCQDMAEPEVFSPNMCQETAVPQTYPPKALEEMAAAEPLSPKMCQETTVSPNHSSKVPQDMAGPEALSPNMCQEPTVPQEHTLKMCHDVARPEVLSPKTHQEMAVPKAFPCVTPGDAAGLEGCAPKALPQSDVAEGCPLDTTPTSVTPEQTTSDPDLGMAVTEGFFSEARECTVSEGVSTKTHQEAVEPEFISHETYKEFTVPIVSSQKTIQESPEPEQYSPETCQPIPGPENYSLETCHEMSGPEDLSIKTCQDREEPKHSLPEGAQKVGGAQGQDADAQDSENAGAFSQDFTEMEEENKADQDPEAPASPQGSQETCPENGIYSSALF

SEQ ID NO: 3 - DNA, 1618 bp, Homo sapiens
  CDS (110)..(1561)
gttatgaagataggtactgtgggtgttagaaagattcacggcaaaacagggaagcatctaggctgcttgtggaagtcagaccaaaatagcaggaaggtattgcagcaagatggatttgggaaaggaccaatctcatttgaagcaccatcagacacctgaccctcatcaagaagagaaccattctccagaagtcattggaacctggagtttgagaaacagagaactacttagaaaaagaaaagctgaagtgcatgaaaaggaaacatcacaatggctatttggagaacagaaaaaacgcaagcagcagagaacaggaaaaggaaatcgaagaggcagaaagagacaacaaaacacagaattgaaggtggagcctcagccacagatagaaaaggaaatagtggagaaagcactggcacctatagagaaaaaaactgagccacctgggagcataaccaaagtatttccttcagtagcctccccgcaaaaagttgtgcctgaggaacacttttctgaaatatgtcaagaaagtaacatatatcaggagaatttttctgagtaccaagaaatagcagtacaaaaccattcttctgaaacatgccaacatgtgtctgaacctgaagacctctctcctaaaatgtaccaagaaatatctgtacttcaagacaattcttccaaaatatgccaagacatgaaggaacctgaagacaactctcctaacacatgccaagtaatatctgtaattcaagaccatcctttcaaaatgtaccaagatatggctaaacgagaagatctggctcctaaaatgtgccaagaagctgctgtacccaaaatccttccttgtccaacatctgaagacacagctgatctggcaggatgctctcttcaagcatatccaaaaccagatgtgcctaaaggctatattcttgacacagaccaaaatccagcagaaccagaggaatacaatgaaacagatcaaggaatagctgagacagaaggcctttttcctaaaatacaagaaatagctgagcctaaagacctttctacaaaaacacaccaagaatcagctgaacctaaataccttcctcataaaacatgtaacgaaattattgtgcctaaagccccctctcataaaacaatccaagaaacacctcattctgaagactattcaattgaaataaaccaagaaactcctgggtctgaaaaatattcacctgaaacgtatcaagaaatacctgggcttgaagaatattcacctgaaatataccaagaaacatcccagcttgaagaatattcacctgaaatataccaagaaacaccggggcctgaagacctctctactgagacatataaaaataaggatgtgcctaaagaatgctttccagaaccacaccaagaaacaggtgggccccaaggccaggatcctaaagcacaccaggaagatgctaaagatgcttatacttttcctcaagaaatgaaagaaaaacccaaagaagagccaggaataccagcaattctgaatgagagtcatccagaaaatgatgtctatagttatgttttgttttaacaatgctcaaccataaagttgtggtccaatggaaaaaaaaaaaaaaaaaaaaaa

SEQ ID NO: 4 - protein, 484 aa, Homo sapiens
MDLGKDQSHLKHHQTPDPHQEENHSPEVIGTWSLRNRELLRKRKAEVHEKETSQWLFGEQKKRKQQRTGKGNRRGRKRQQNTELKVEPQPQIEKEIVEKALAPIEKKTEPPGSITKVFPSVASPQKVVPEEHFSEICQESNIYQENFSEYQEIAVQNHSSETCQHVSEPEDLSPKMYQEISVLQDNSSKICQDMKEPEDNSPNTCQVISVIQDHPFKMYQDMAKREDLAPKMCQEAAVPKILPCPTSEDTADLAGCSLQAYPKPDVPKGYILDTDQNPAEPEEYNETDQGIAETEGLFPKIQEIAEPKDLSTKTHQESAEPKYLPHKTCNEIIVPKAPSHKTIQETPHSEDYSIEINQETPGSEKYSPETYQEIPGLEEYSPEIYQETSQLEEYSPEIYQETPGPEDLSTETYKNKDVPKECFPEPHQETGGPQGQDPKAHQEDAKDAYTFPQEMKEKPKEEPGIPAILNESHPENDVYSYVLF

SEQ ID NO: 5 - DNA, 1455 bp, Homo sapiens
atggatttgggaaaggaccaatctcatttgaagcaccatcagacacctgaccctcatcaagaagagaaccattctccagaagtcattggaacctggagtttgagaaacagagaactacttagaaaaagaaaagctgaagtgcatgaaaaggaaacatcacaatggctatttggagaacagaaaaaacgcaagcagcagagaacaggaaaaggaaatcgaagaggcagaaagagacaacaaaacacagaattgaaggtggagcctcagccacagatagaaaaggaaatagtggagaaagcactggcacctatagagaaaaaaactgagccacctgggagcataaccaaagtatttccttcagtagcctccccgcaaaaagttgtgcctgaggaacacttttctgaaatatgtcaagaaagtaacatatatcaggagaatttttctgagtaccaagaaatagcagtacaaaaccattcttctgaaacatgccaacatgtgtctgaacctgaagacctctctcctaaaatgtaccaagaaatatctgtacttcaagacaattcttccaaaatatgccaagacatgaaggaacctgaagacaactctcctaacacatgccaagtaatatctgtaattcaagaccatcctttcaaaatgtaccaagatatggctaaacgagaagatctggctcctaaaatgtgccaagaagctgctgtacccaaaatccttccttgtccaacatctgaagacacagctgatctggcaggatgctctcttcaagcatatccaaaaccagatgtgcctaaaggctatattcttgacacagaccaaaatccagcagaaccagaggaatacaatgaaacagatcaaggaatagctgagacagaaggcctttttcctaaaatacaagaaatagctgagcctaaagacctttctacaaaaacacaccaagaatcagctgaacctaaataccttcctcataaaacatgtaacgaaattattgtgcctaaagccccctctcataaaacaatccaagaaacacctcattctgaagactattcaattgaaataaaccaagaaactcctgggtctgaaaaatattcacctgaaacgtatcaagaaatacctgggcttgaagaatattcacctgaaatataccaagaaacatcccagcttgaagaatattcacctgaaatataccaagaaacaccggggcctgaagacctctctactgagacatataaaaataaggatgtgcctaaagaatgctttccagaaccacaccaagaaacaggtgggccccaaggccaggatcctaaagcacaccaggaagatgctaaagatgcttatacttttcctcaagaaatgaaagaaaaacccaaagaagagccaggaataccagcaattctgaatgagagtcatccagaaaatgatgtctatagttatgttttgttttaa

SEQ ID NO: 6 - DNA, 22 bp, Artificial Sequence
  Primer
aaacacacctctctcctaccac

SEQ ID NO: 7 - DNA, 22 bp, Artificial Sequence
  Primer
cctactttctgggctccttctg

SEQ ID NO: 8 - DNA, 22 bp, Artificial Sequence
  Primer
aagcaccatcagacacctgacc

SEQ ID NO: 9 - DNA, 22 bp, Artificial Sequence
  Primer
tgcttgaagagagcatcctgcc

SEQ ID NO: 10 - DNA, 20 bp, Artificial Sequence
  Primer
ccactgaacttctgattcgc

SEQ ID NO: 11 - DNA, 20 bp, Artificial Sequence
  Primer
gggtgctagctggatgtctt